(19) United States
(12) Patent Application Publication
(10) Pub. No.: US 2010/0121201 A1
(43) Pub. Date: May 13, 2010

(54) Non-invasive wound prevention, detection, and analysis

(76) Inventor: George Yiorgos Papaioannou, Glendale, WI (US)

Correspondence Address: MICHAEL BEST & FRIEDRICH LLP, 100 E WISCONSIN AVENUE, Suite 3300, MILWAUKEE, WI, 53202, US

(21) Appl. No.: 12/587,831

(22) Filed: Oct. 13, 2009

Related U.S. Application Data
(60) Provisional application No. 61/104,968, filed on Oct. 13, 2008.

(51) Int. Cl. A61B 6/00; G06K 9/00

(52) U.S. Cl. 600/477; 382/128; 600/476; 382/154; 382/165

(57) ABSTRACT

A computer-based method of analyzing a wound. An image of the wound is captured by a camera and a three-dimensional model of the wound is generated based on the image. A volume of the wound is calculated based on the three-dimensional model and changes to the calculated volume are monitored over a period of time.

### RELATED APPLICATIONS

The present application claims priority to U.S. Provisional Application No. 61/104,968 filed on Oc. 13, 2008, the entire contents of which is incorporated herein by reference.

### BACKGROUND

The present invention relates generally to the analysis of wounds and, more particularly, to methods and systems for minimally invasive analysis and monitoring of wounds such as pressure ulcers or skin burns.

Pressure ulcers can occur when a person applies force to an area of the skin for an extended period of time—for example, a patient who is confined to a therapy bed while recovering from an injury or a paraplegic who uses a wheelchair. It is estimated that 85% of spinal cord injured patients that utilize a wheelchair will develop a pressure ulcer during their lifetime. Pressure ulcers or similar wounds can also occur when a skin surface is exposed to repetitive forces—for example, persons fitted with prosthetic devices. Pressure ulcers (and similar skin wounds such as toxic or heat burns, skin macerations, or amputations) can lead to infections if not properly monitored and treated.

The likelihood of a pressure ulcer developing is influenced by factors such as the magnitude, duration, direction, and distribution of the load applied to the skin surface. Risk assessment scales have been developed that use such factors to calculate a score indicative of a patient's risk of developing a pressure ulcer. Some such risk assessment scales include the Norton scale, the Braden scale, the Waterlow scale, and variations thereof.

After a wound, such as a pressure ulcer, has developed, it will tend to close first from its base rather than from its edge. As such, the monitoring of the early-stage healing process focuses on wound depth and wound volume rather than wound area. The most widely used methods for volumetric measurements of a wound currently involve filling the wound with saline or creating an alginate mold of the wound. However, such techniques are uncomfortable and painful to the patient and can lead to infection.

### SUMMARY

Various embodiments of the invention provide camera-based systems and methods for capturing digital wound data and calculating wound statistics including area, volume, depth, and color. The system uses these statistics, digital skin mapping, and other patient data to evaluate existing wounds and determine the risk of developing new wounds. Because the system is camera-based, the system and methods of the invention are minimally invasive and reduce the discomfort and risk of infection to the patient.

In one embodiment, the invention provides a computer-based method of analyzing a wound. An image of the wound is captured by a camera and a three-dimensional model of the wound is generated based on the image. A volume of the wound is calculated based on the three-dimensional model and changes to the calculated volume are monitored over a period of time.

In some embodiments, several parallel light lines are projected on the wound from a light source that is located at an angle relative to the camera. The method then generates the three-dimensional model of the wound by identifying individual light lines and estimating the location of data points along each light line in a three-dimensional space using triangulation based on the angle of the camera relative to the light source.

In some embodiments, a grid of light lines is projected on the wound. The grid includes several horizontal light lines and several vertical light lines positioned perpendicular to the horizontal lines. The three-dimensional model of the wound is then generated by identifying a plurality of intersection points between the horizontal lines and the vertical lines. The location of each intersection point in a three-dimensional space is estimated based on a known distance between each intersection point from the angle of the light source.

In some embodiments, a light source scans a single light line across the surface of the wound while a camera captures multiple pictures of the wound. The three-dimensional model of the wound is then generated by estimating the location of data points on the single light line in each of the pictures. The data points from each of the pictures are then incorporated into a single three-dimensional model.

In another embodiment, the invention provides a wound analysis system that includes a light source and a first camera. The light source is positioned to project at least one light line on a wound and the first camera is positioned to capture a first image of the wound at an angle relative to the light source. The system also includes an image processing system that accesses the first image, generates a three-dimensional model of the wound based on the first image, calculates a volume of the wound based on the three-dimensional model, and monitors changes to the calculated volume of the wound over a period of time.

Other aspects of the invention will become apparent by consideration of the detailed description and the drawings.

### BRIEF DESCRIPTION OF THE DRAWINGS AND APPENDICES

FIG. 1 is a schematic, overhead view of a wound analysis system according to one embodiment.

FIG. 2 is a perspective view of light lines projected on a wound surface according to one embodiment of the system of FIG. 1.

FIG. 3 is a flowchart illustrating a method for generating a three-dimensional model of an image that includes parallel light lines projected on the target wound as illustrated in FIG. 2.

FIG. 4a is a perspective view of a target object (in the case illustrated, a human hand) with a grid pattern projected on the object from a light source according to another embodiment of the system of FIG. 1.

FIG. 4b is a perspective view of a target object with a single light line projected on the object from a light source according to another embodiment of the system of FIG. 1.

FIG. 5 is a flowchart illustrating a method for generating a three-dimensional model of a wound from a plurality of images of the wound each including a single light line projected at a different location on the wound.

FIGS. 6a, 6b, and 6c are digital reconstructions of the 3-D surface of a wound generated by the system of FIG. 1.

FIG. 7 is a perspective view of the system arrangement and calibration equipment used when calibrating a two-camera stereophotogrammetery-based wound analysis system of FIG. 1.

FIG. 8 is a flowchart showing the operation of the wound analysis archiving system from patient admittance through report generation.

FIG. 9 is an image of a screen from a graphical user interface of the wound analysis system showing an image of the wound, a single color histogram, and a 3D graph of color density.

FIGS. 10a and 10b are graphical representations of the volumes associated with the “wound volume” statistic calculated by the wound analysis system of FIG. 1.

FIG. 11a is a perspective view of the system of FIG. 1 with a wounded limb positioned in front of the camera.

FIG. 11b is another perspective view of the wounded limb.

FIGS. 11c-11g are three-dimension models of the limb wound generated by the system of FIG. 1.

FIG. 12 is an image of a screen from a graphical user interface of the wound analysis system displaying wound color and shape information over time.

FIG. 13 is an image of a screen from a graphical user interface of the wound analysis system displaying additional patient and wound diagnosis information.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

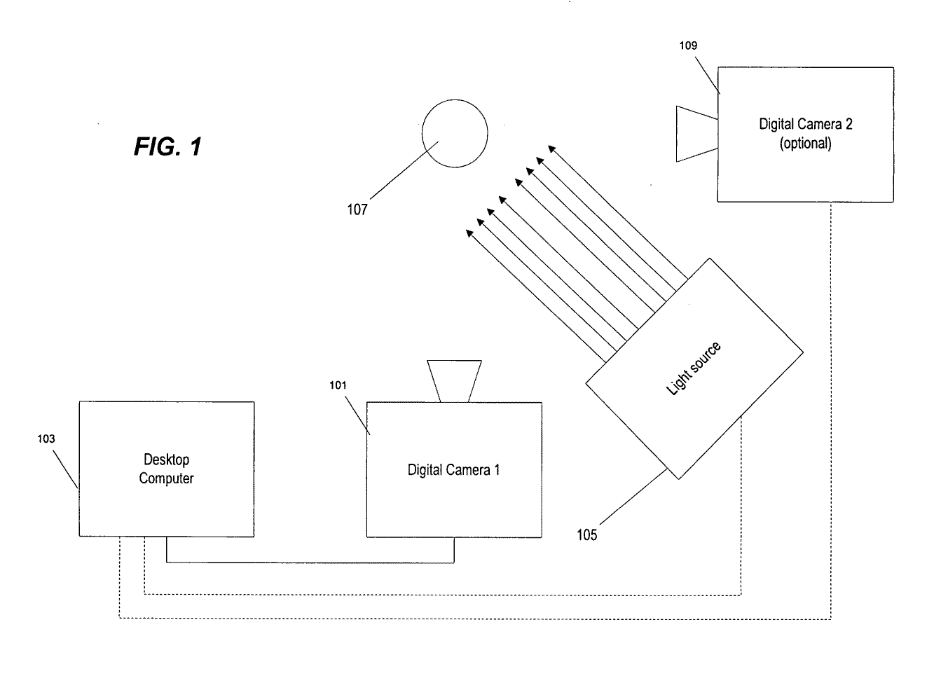

FIG. 1 shows the interconnections and layout of hardware components according to one embodiment of the wound analysis system. At least one digital camera 101 is connected to a desktop computer 103. A light source 105 is also provided. The light source 105 is configured to project a plurality of parallel planar light beams toward a target object 107. When the light beams strike the target 107, a series of parallel light lines are projected on the target object 107. The digital camera 101 is positioned to capture an image of the target object 107, but at an angle different than that of the light source 105. As described in detail below, in some embodiments, a second digital camera 109 may be used to provide further detail for the digital reconstruction of certain types of wounds.

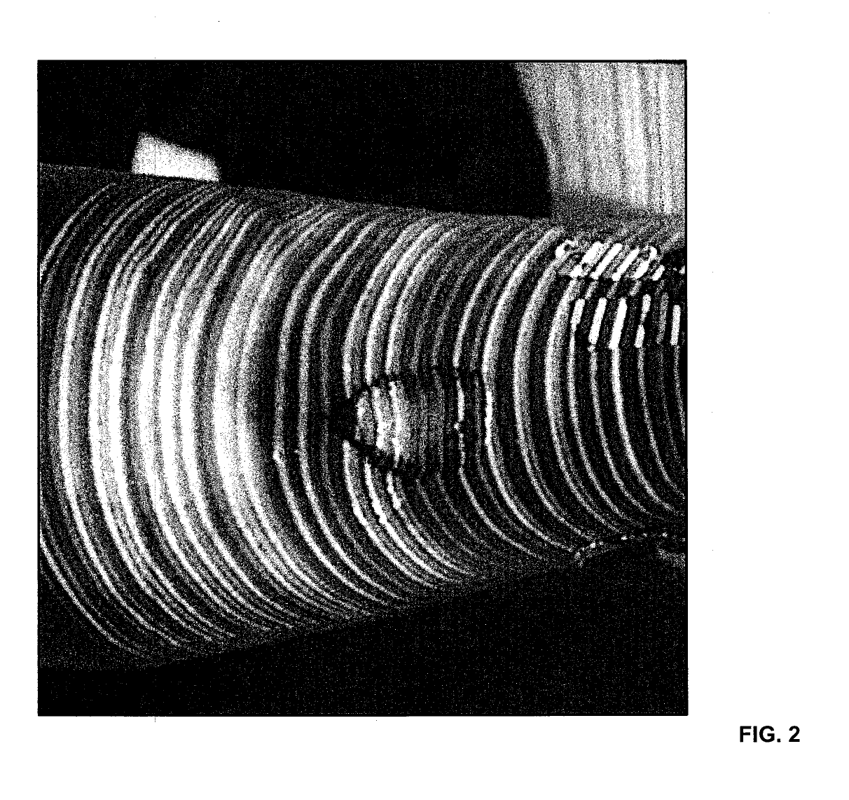

The light source 105 in the example of FIG. 1 is connected to the desktop computer 103. The desktop computer 103 controls the light source 105 by sending control instructions to the light source 105. However, in some embodiments, the light source 105 is operated separately from the desktop computer 103. The light source 105 in the embodiment shown in FIG. 1 (and later in FIG. 2) includes one or more light bulbs positioned behind a mask. The mask divides the light from the bulbs into a series of perpendicular planar light beams. A series of colored light filters or gels is incorporated into the mask to alter the color of each planar light beam. The result, as shown in FIG. 2, is that a series of parallel light lines 201 are projected on the target object 107. Adjacent projected light lines are of different colors so that the imaging system running on the desktop computer 103 is able to distinguish between the lines during digital reconstruction of the target object 107.

In other embodiments, the light source can include a variety of other light emission arrangements such as, for example, a series of various colored lasers or light emitting diodes. Although the projected light lines in this embodiment are each of a different color, in other embodiments, the project light lines can be the same color, alternating colors, or other combinations of single or multiple colors.

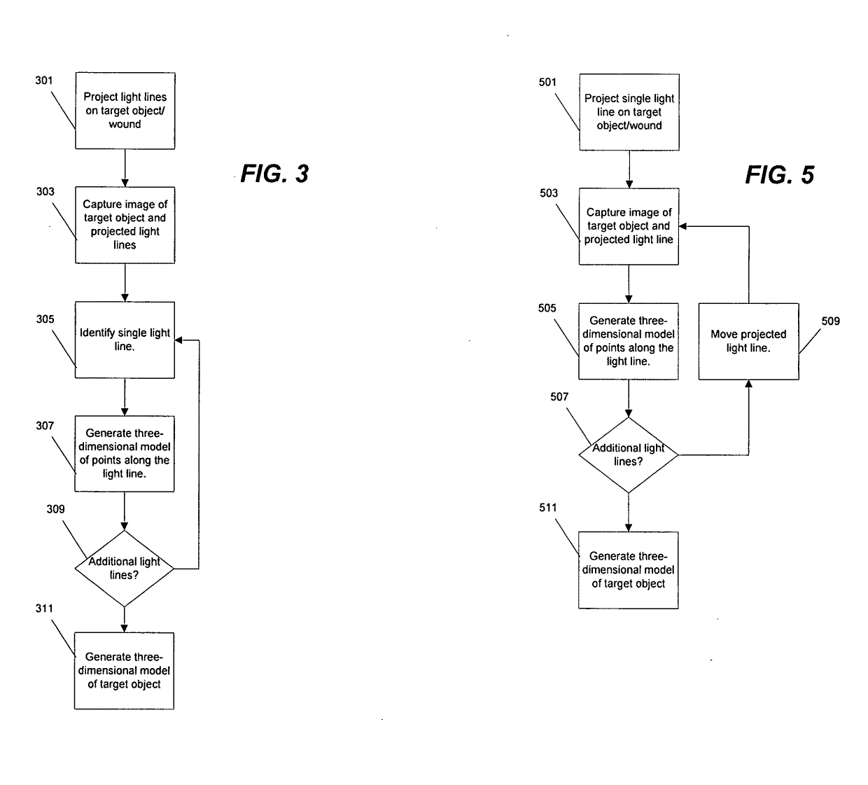

The desktop computer 103 then generates a three-dimensional model of the target object. FIG. 3 illustrates one example of a method of generating such a three-dimensional model. The light source 105 projects a series of parallel light lines on the wound (step 301). The digital camera 103 captures an image of the wound with the projected light lines (step 303). The image is sent to the desktop computer 103 and the image processing system running on the desktop computer 103 identifies a first light line in the image (step 305).

The image processing system then generates a three-dimensional model of the points along the light line (step 307). This can be accomplished using triangulation techniques. For example, the image processing system can assume that the image of the projected light lines will be parallel from the perspective of the light source 105. The image processing system can then perform triangulation of the points along the project light lines based on the angle of the light source 105 relative to the digital camera 101.

The image processing system repeats this reconstruction for each light line in the captured image (step 309). The system then generates a three dimensional model of the wound by incorporating the data points for each individual line into a single model representation. Data points from adjacent light lines are connected in the final three-dimensional model to complete the modeled surface of the wound. As such, the accuracy of the modeling system is increased by increasing the number of light lines that are projected on the wound.

Some wounds will be of sufficient depths that portions of the projected light line in the image captured by the first digital camera 101 will be obscured or completely blocked by the wound itself. As such, the second digital camera 109 can be used to capture an image of the wound and the projected light lines from a different angle. Several known methods of three-dimensional image modeling can be used to reconstruct a three-dimensional model of the wound from two two-dimensional images using stereophotogrammetry including the PhotoModeler software package (produced by EOS Systems, Inc., Vancouver, Canada).

The triangulation procedure described above is then used to generate a three-dimensional model of the wound as observed by the second camera 109. The two three-dimensional models are then combined to create a single three-dimensional model that includes data points for all surfaces of the wound.

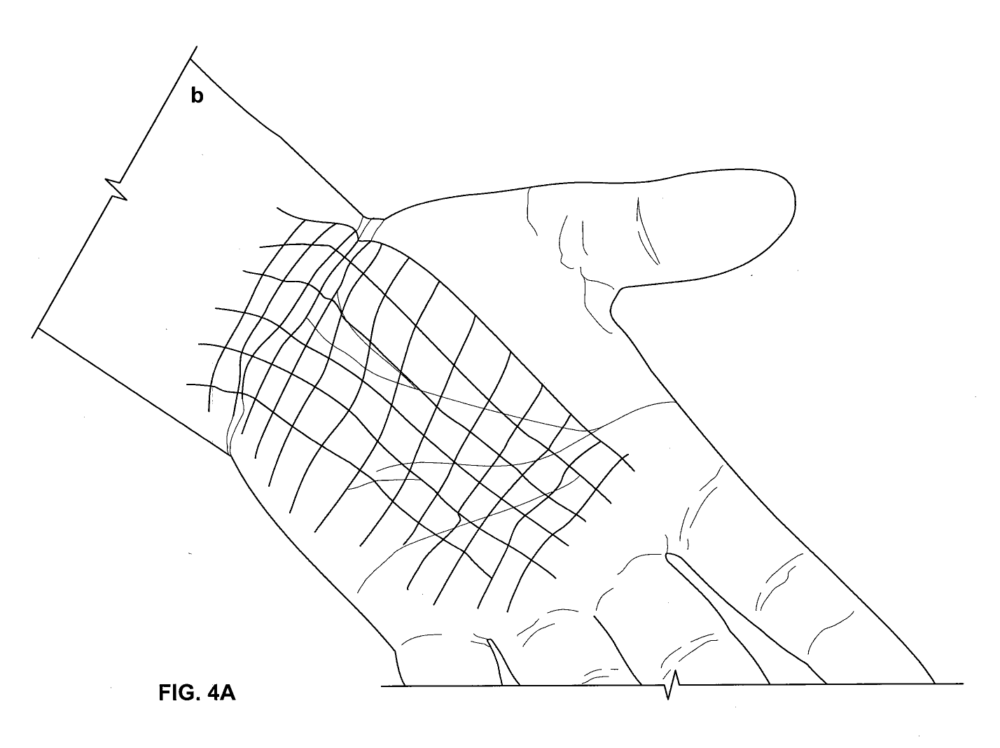

Other similar imaging techniques can also be used to generate an image of the wound. For example, as illustrated in FIG. 4a, the light source 105 can project a grid pattern on the target object 105 instead of only project parallel light lines. The grid pattern provides additional data points that can be located by the image processing system and included in the three-dimensional model. The grid pattern can also be helpful in simplifying the computational requirements of the image process. Instead of approximating the location of multiple points along each intersection line in the grid, the image processing system in some embodiments can approximate the location of each intersection point on the grid using triangulation and the known distance between each projected intersection.

In yet another embodiment, the light source does not project a series of parallel lines across the target object. Instead, as illustrated in FIG. 4b, the light source projects a single light line 403 on the target object 401. The light source then moves the light line 403 in a parallel direction across the surface of the wound. The image processing system in this embodiment uses the same type of triangulation to identify the location of data points in the wound images. However, instead of differentiating between parallel lines projected on the surface of the wound, the image processing system receives a series of images that each include only a single projected light line in different locations.

FIG. 5 illustrates a method of generating the three-dimensional model of the wound using this type of light source. The light source 105 projects the single light line on the wound 501 (step 501). The digital camera 101 captures an image of the wound and the projected light line (step 503). The image is sent to the image processing system running on the desktop computer 103 and a three-dimensional model is generated of the points along the single light line (step 505). If more data is required to generate a model of the entire wound (step 507), the light source 105 moves the projected light lines to a different location on the wound (step 509), the digital camera 101 captures a new image of the projected light line on the wound (step 503), and the image processing system generates another set of data points (step 505). When data points have been captured for the entire wound area, the image processing system generates the three-dimensional model of the target object (step 511).

Although a desktop computer is used for the image processing in the above examples, other embodiments may include other data processing units. For example, in some embodiments, the digital camera 101, the light source 105, and a dedicated data processing unit are integrated into a single unit housing. In other embodiments, the digital camera 101 captures an image of the light lines projected on the target object 107 and sends the image to a remote computer system to be processed and analyzed.

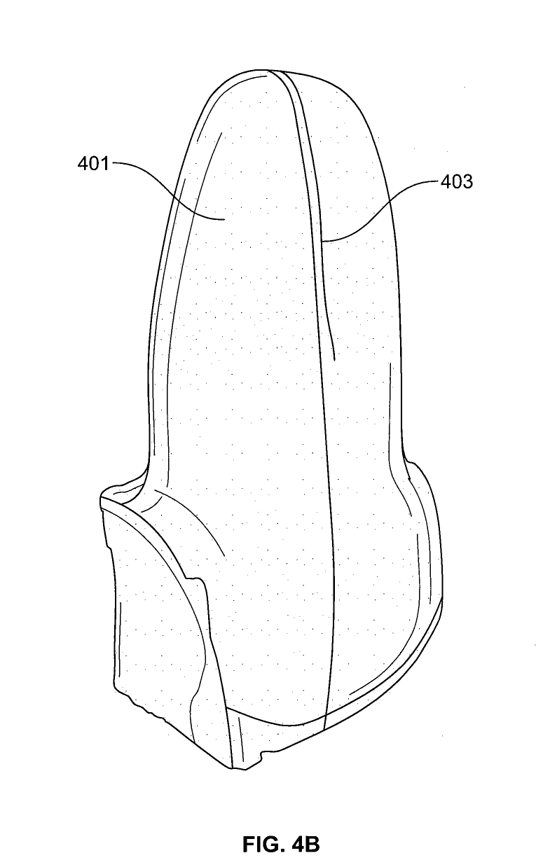

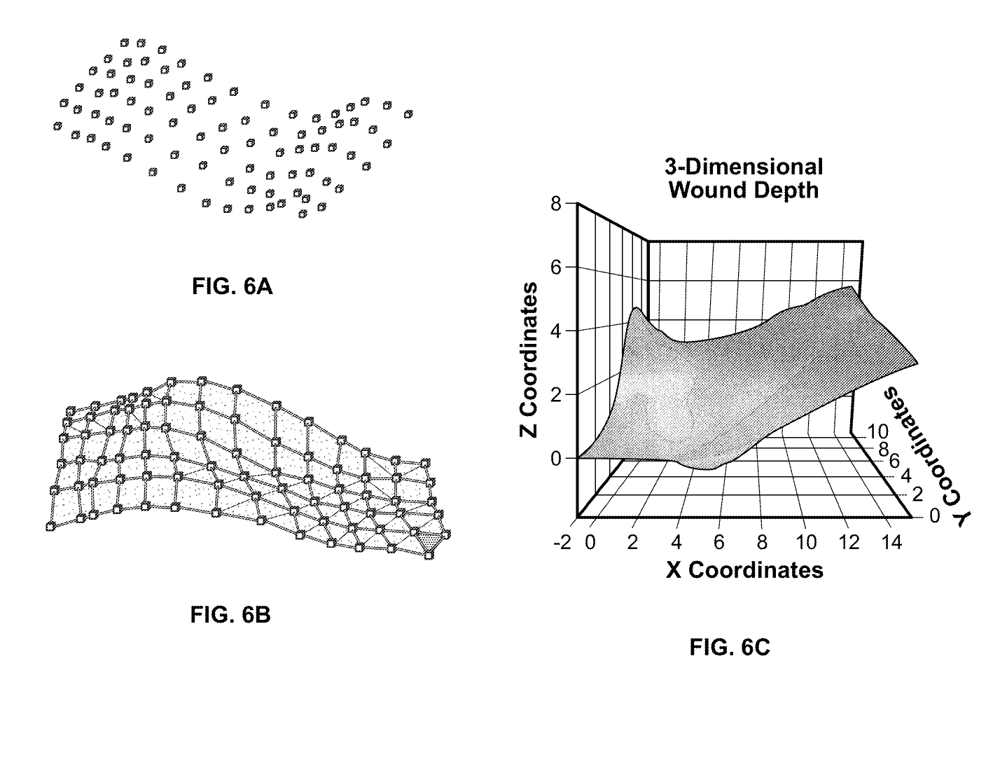

FIG. 6 illustrates several examples of the three-dimensional model generated by the image processing system. FIG. 6a shows several data points identified from the captured images. In FIG. 6b, the data points from adjacent lines are connected to form a completed surface for the three-dimensional model. In FIG. 6c, the lines between adjacent points are smoothed to estimate the actual surface of the wound.

The digital cameras 101 and 109 described above can be almost any model with sufficient resolution. For example, a Nikon D2Xs with a Nikon AF-S Micro Nikkor 105 mm lens and a Nikon Close-up Speedlight kit with one SU-800 Wireless Speedlight Commander and two SB-200 Wireless Remote Speedlights can be used. Alternatively, a simple, commercially available camera system such as the Canon PowerShot A80 can be used as the primary camera 101 or the secondary camera 109.

The camera arrangement can be calibrated by direct linear transformation (DLT). In DLT, space is calibrated by capturing images of an object of known dimensions. These dimensions can later be used to map the position of portions of the wound. Various other camera calibration methods can alternatively be used, such as disclosed in Keikkila, J. et al., A Four-step Camera Calibration Procedure with Implicit image Correction, IEEE Computer Society Conference on Computer Vision and Pattern Recognition; 1997 (pp. 1106-1112), the entire contents of which are incorporated herein by reference.

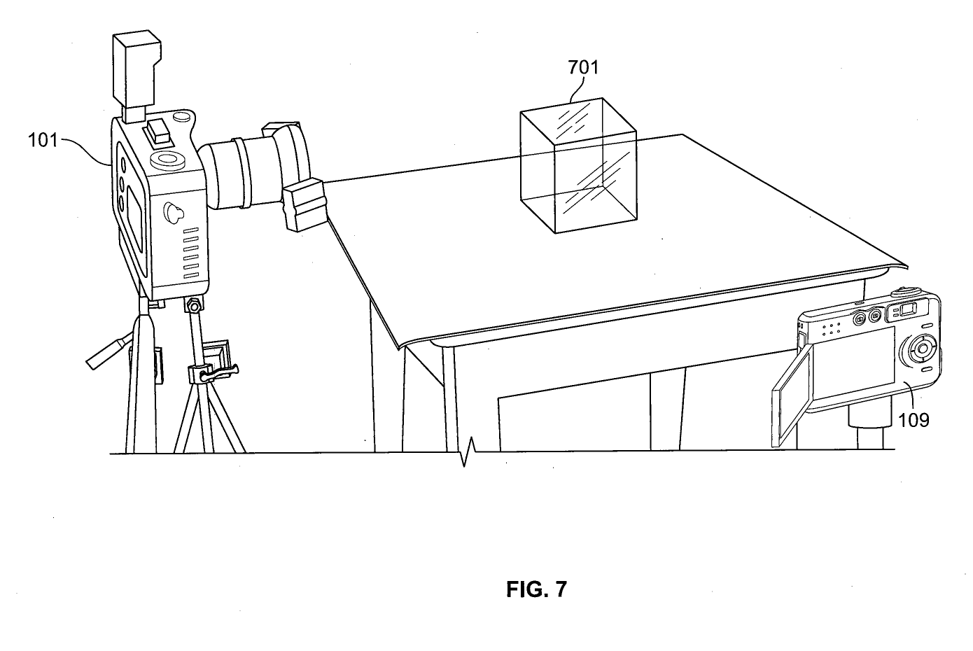

FIG. 7 illustrates the calibration set-up for the imaging system described above. The two cameras 101, 109 are mounted 0.5 m from the center of a calibration object 701 placed in an imaging volume. The cameras 101, 109 are pointed toward the calibration object 701 at 90° relative to each other. The calibration object 701 is a transparent cube. The calibration object 701 is then repositioned by rotating on all three axis and a multiple pairs of images are captured by the cameras, each of a different orientation of the calibration object. A minimum of five pairs of calibration images has been found to improve the quality of fit of the resulting three-dimensional modeling; however, using ten pairs of images has been found to provide an even better quality of fit.

In addition to spatial calibration (using a calibration object of known dimensions 701), color calibration is performed using a RGB color sample of known color intensities. The RGB color sample is placed in the imaging volume near the calibration object 701. An image is captured by each camera and used as a reference during the image analysis discussed below.

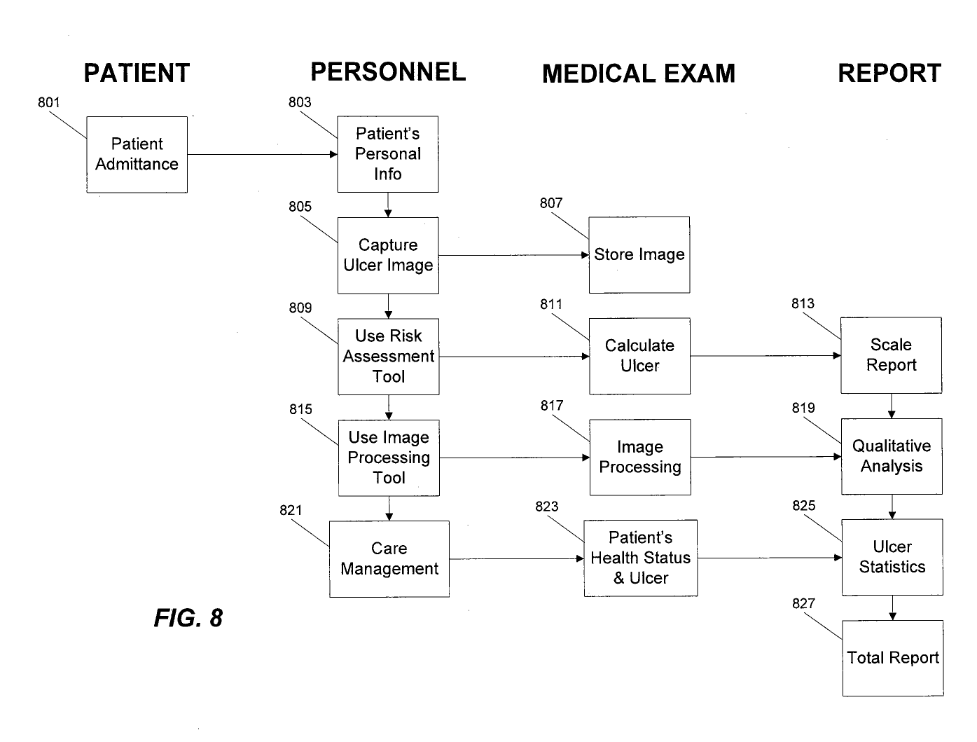

FIG. 8 provides an overview of the logical operations performed by the wound analysis system. After a patient is admitted (step 801), the patient's personal information and medical history data is entered or accessed from a system database (step 803). An image of the wound is captured (step 805) and stored locally or to the hospital's picture archive and communications system (PACS) (step 807). The image of the wound is sent to a risk assessment tool (described below) (step 809) which calculates information about the ulcer (step 811) and produces a scale report (step 813). The image is then sent to the image processing tool described above (step 815). In addition to generating the three-dimensional model of the wound, the image processing tool processes the image (step 817) and produces a qualitative analysis including information regarding size, color, and volume of the wound (step 819). A care management tool is then accessed (step 821) which examines the patient's health status (step 823) and produces an ulcer statistics report (step 825) to be used by the healthcare professional when determining an appropriate course of treatment. The scale report, the qualitative image analysis, and the ulcer statistics are then combined to generate a full report (step 827).

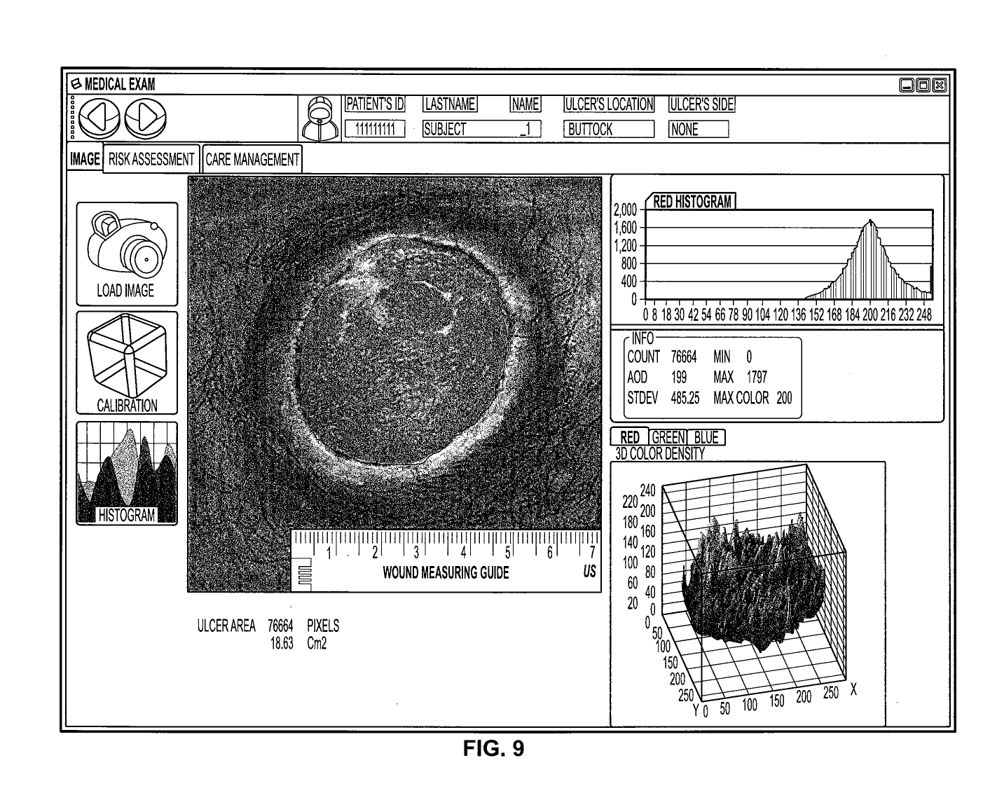

FIG. 9 shows a screen image presented on the monitor of the desktop computer in FIG. 1. In other embodiments that do not utilize a desktop computer, a similar screen shot is presented on a graphical user interface incorporated into the wound analysis system. The screen shows an image of the wound captured by one of the digital cameras. The image includes the wound and a measuring guide to provide a reference scale for the user. To further aid the three-dimensional modeling, the interface of the screen shown in FIG. 9 allows the user to trace the edge of the image of the ulcer with a curser controller by a mouse or stylus. The data processing unit will then confine its statistical analysis and three-dimensional modeling to areas within the traced range. Therefore, the system and the user are able to disregard data related to the skin surface and focus on the wound itself. Some embodiments include logic for approximating the edge of the wound based on changes in color or height as observed in the wound images.

After the user has defined the edge of the wound area, the wound analysis system is able to begin its statistical analysis of the wound. As shown beneath the wound image on the screen of FIG. 8, the area of the ulcer opening is calculated and displayed in both pixels and cm². The wound analysis system then uses an RGB color model to determine the color density of each pixel in the wound area. A histogram (upper right of FIG. 8) is generated that displays the number of pixels associated with each level of color density. A 3D color density graph shows the color density of each pixel plotted against the two-dimensional surface of the wound. In this embodiment, the 3D color density graph does not account for the three-dimensional shape of the wound itself. The graphical user interface of the screen shown in FIG. 8 includes tabs that allow the user to select whether to display statistical data for red, green, or blue. The screen also displays patient information including a patient ID number, last name, first name, ulcer location, and ulcer side.

In some embodiments, the wound analysis system uses the same wound images for reconstructing a three-dimensional model of the wound as it does for color analysis. In other embodiments, an image of the wound is captured perpendicular to the wound surface. The image is then processed to create an orthophoto. An orthophoto is an image in which all perspective related distortions have been removed. The orthophoto is then used for color analysis as described below.

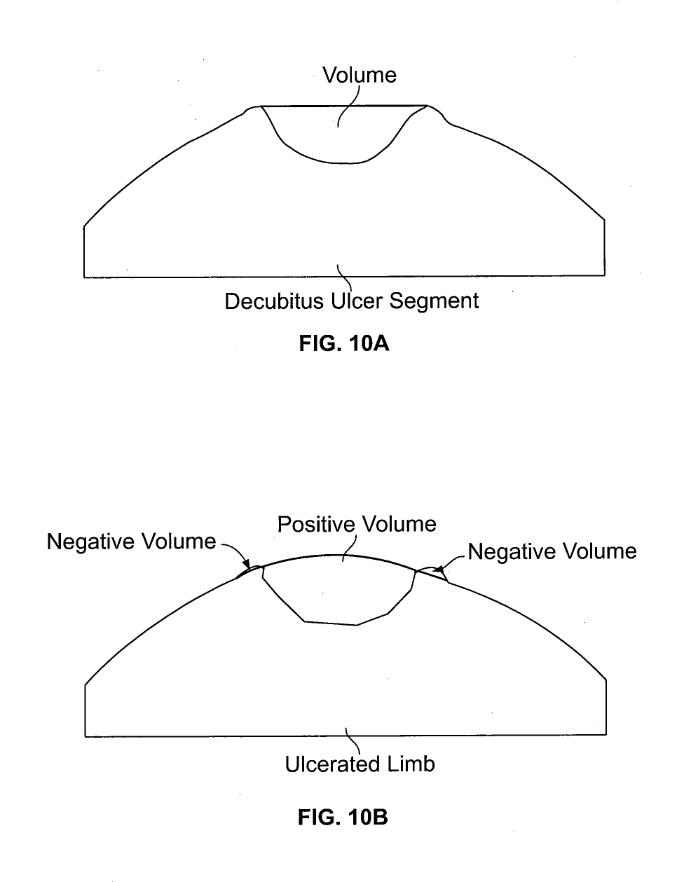

Three-dimensional digital models of the wound constructed by the system provide a non-invasive mechanism for calculating the depth and volume of the wound. However, wound volume is not subject to a single, universally-accepted standard. In fact, it is defined differently under different standards and techniques. FIGS. 10a and 10b illustrate two possible standards to be used in calculating wound volume. When a pressure ulcer (or another type of wound) forms, the area around the wound may become slightly raised. Traditionally, a healthcare provider measures wound volume by filling the wound to the top of the raised edge portion (as shown in FIG. 9a). Therefore, in some embodiments of the wound analysis system, the volume of the wound is calculated as the volume between the three-dimensional digital model of the wound and a plane that contacts the raised wound edge.

In other embodiments, the wound analysis system approximates the normal shape of the skin as if the wound had not occurred or when the wound is fully healed. As shown in FIG. 9b, the negative volume associated with the raised edge is removed from the digitally-constructed, three-dimensional model of the wound, and an approximate skin level is determined based on the shape of the skin surrounding the wound beyond the raised edge. The positive volume of the wound is then calculated as the volume between the three-dimensional digital model of the wound and the three-dimensional approximation of what the skin shape would be if the wound was not present.

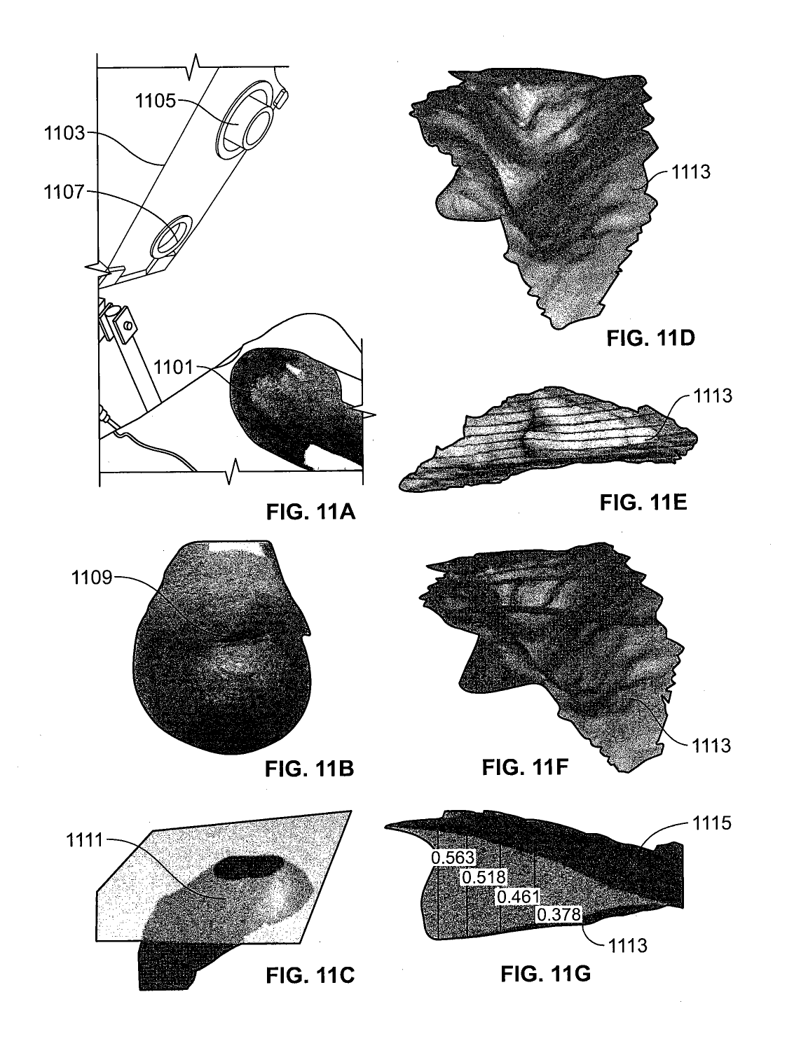

FIG. 11 illustrates the methods of three-dimensional modeling and the analysis of the three-dimensional model as described above. FIG. 11a shows a patient limb 1101 with a pressure ulcer placed in front of a digital camera system 1103. The digital camera system 1103 includes a digital camera 1105 and a light source 1107. The light source 1107 emits a single planar laser beam that projects a light line on the patient limb. FIG. 11b shows the pressure ulcer 1109 on the patient limb 1101 from a different angle.

FIG. 11c shows the initial three-dimensional model 1111 of the wounded limb 1101. FIGS. 11d, 11e, and 11f show different perspectives of a three-dimensional model 1113 of the wound volume as isolated from the rest of the limb. In FIG. 11g, the image processing system has estimated the geometry of a healthy limb 1101 based on the geometry of the skin surrounding the wound. A estimated three-dimensional model 1115 of the healthy limb is superimposed over the three-dimensional model 113 of the wound. The volume between the two models 1113, 1115 is then calculated by the image processing system.

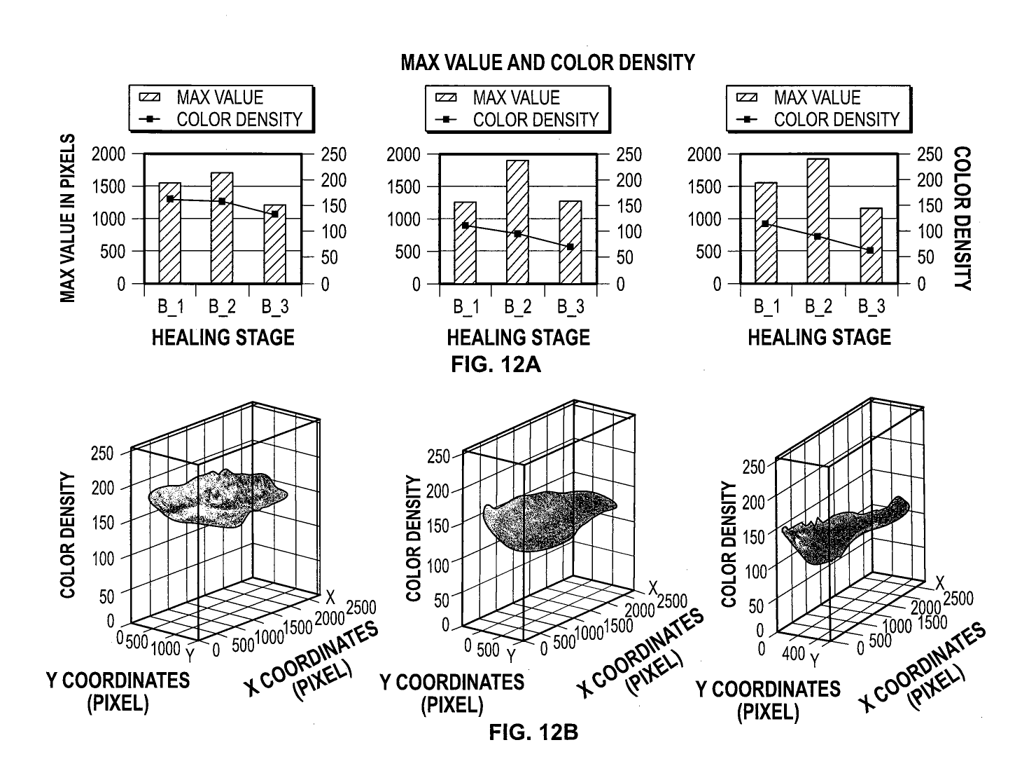

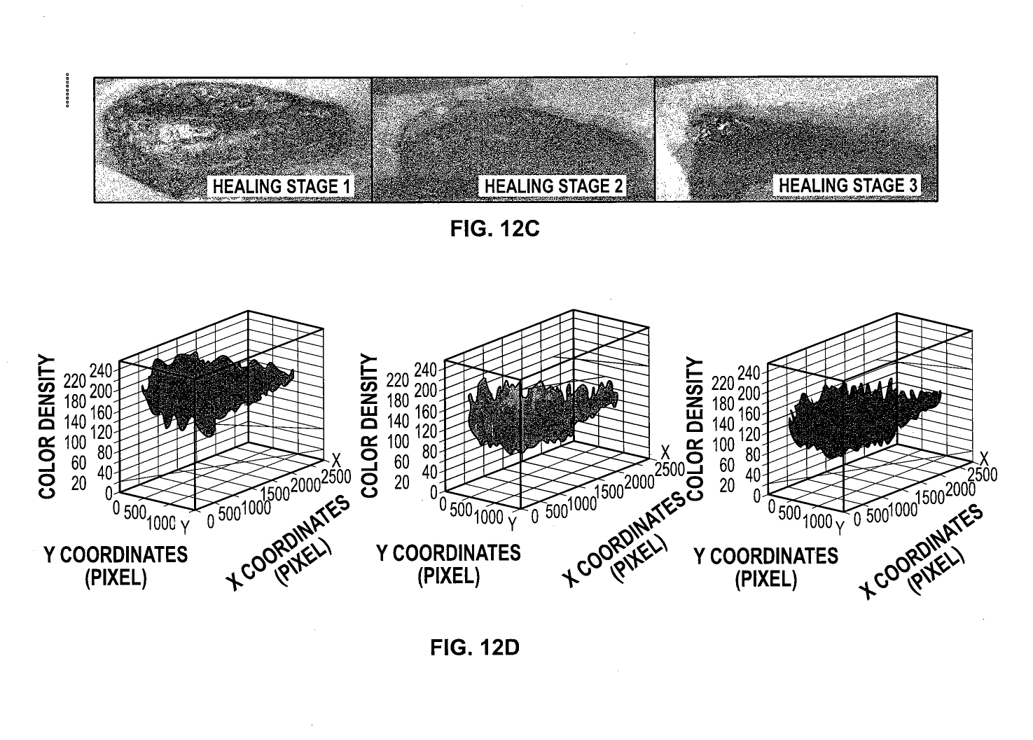

One benefit of the wound analysis system is the ability to track changes in the wound over a period of time. FIG. 12 shows another screen displayed to the user on the desktop computer 103. The top row (Row I) provides three graphs each associated with a different color in the RGB color model (the far left is red, the center is green, the far right is blue). Each graph in the top row includes a series of three bars and three dots. Each set (i.e., one dot and one bar) corresponds to a wound image captured at a different stage of recovery (e.g., images captured at three different times). The dots on each graph correspond to the color density value (as indicated on the right hand scale of the graph) that was observed in the greatest number of pixels (i.e., the bin of the color histogram with the most pixels). The bar elements correspond to the number of pixels that are associated with that color density value.

Color density value is one way that the wound analysis system quantifies the healing of the wound. As the wound heals, extreme red, green, or blue colors begin to fade as the color of the wound area returns to a flesh color. Therefore, the color density that is observed in the greatest number of pixels is a lower color density value as the wound heals. If the color density value displayed on the graph does not decline over time (or if the decline is not as rapid as it should be), the healthcare provider can use this information to recommend a different course of treatment.

The second row (Row II) on the graph shows three reconstructed three-dimensional models of the wound. Each model was created using data from images captured at a different stage of the healing process. The three three-dimensional models displayed together allow the patient and the healthcare provider to view and analyze how the shape of the wound has changed over time. Each three-dimensional model is colored according to the color density information retrieved from the respective image. If the shape and volume of the wound is not decreasing (or is not decreasing rapidly enough), the healthcare provider can use this information to recommend a different course of treatment.

The third row (Row III) shows photographic images of the wound same wound area over the course of treatment (such as the wound image shown in FIG. 8. The bottom row (Row IV) shows a 3D color density graph for each color (red, green, and blue) exhibited in the wound image during the same healing stage.

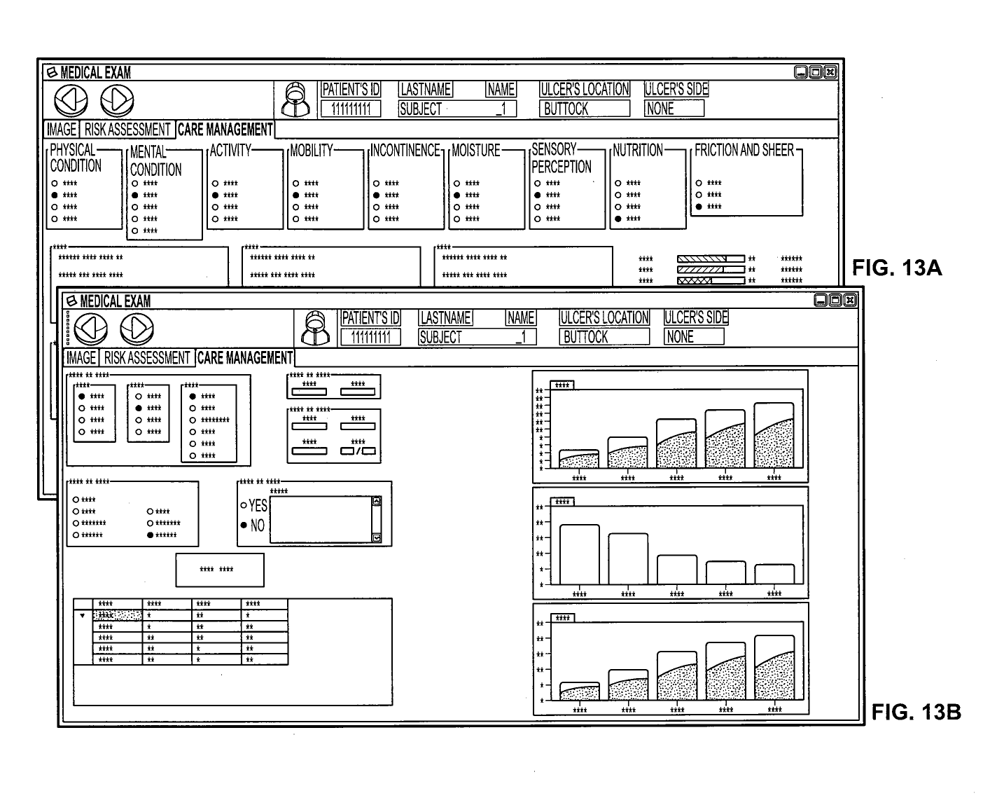

Returning to the graphical user interface of FIG. 9, three tabs are presented at the top of the screen. The tabs are labeled “Image,” “Risk Assessment,” and “Care Management.” FIG. 9 shows the graphical user interface when the “Image” tab is selected. FIGS. 13a and 13b show the graphical user interface when the other two tabs are selected, respectively. The Care Management tab of FIG. 13b displays a variety of information related to the treatment of the patient including vital signs, fluid balance, and patient history.

On the Risk Assessment page of FIG. 13a, the patient or the health care professional can select wound condition details and answer a plurality of questions related to the patient's health and skin condition. Data collected in this manner can include, for example, physical condition of the patient, mental condition of the patient, mobility, activity, incontinence, sensory perception, moisture of the surface, nutrition, friction, and sheer forces. The data processing unit uses this information and statistical information derived from the image analysis described above to calculate a risk score according to one or more risk assessment scales (RAS). A risk score approximates the likelihood of a given patient developing a pressure ulcer. Commonly used RASs include the Norton scale, the Braden scale, the Waterlow scale, and the Gosnell scale. As the user enters or changes information on this page, all of the risk scores change simultaneously. The risk scores can be analyzed at the time of calculation and can be compared to previously stored risk scores to monitor changes in risk over time. All such information is stored in electronic reports that can be distributed over the Internet or through hospital information systems (e.g., Oracle- or SQL-based systems).

The non-invasive data capture technology and the wide array of statistical computation and display capabilities of the various embodiments of the wound analysis system provide for comprehensive and easy to use wound prevention, management, and analysis systems. Various features and advantages of the invention are set forth in the drawings and claims.

What is claimed is:

1. A computer-based method of analyzing a wound, the method comprising:
capturing an image of the wound;
generating a three-dimensional model of the wound;
calculating a volume of the wound based on the three-dimensional model; and
monitoring changes to the calculated volume of the wound over a period of time.

2. The computer-based method of claim 1, further comprising projecting a plurality of parallel light lines on the wound from a light source located at an angle from the wound relative to a camera that captures the image of the wound, and wherein generating a three-dimensional model of the wound includes
identifying a first light line from the plurality of light lines, and
estimating the location of a plurality of data points along the first light line in a three-dimensional space by triangulation based on the angle of the camera relative to the light source.

3. The computer-based method of claim 1, further comprising projecting a grid of light lines on the wound from a light source located at an angle from the wound relative to a camera that captures the image of the wound, the grid of light lines including a plurality of horizontal lines and a plurality of vertical lines positioned perpendicular to the plurality of horizontal lines.

4. The computer-based method of claim 3, wherein generating a three-dimensional model of the wound includes
identifying a plurality of intersection points between the horizontal lines and the vertical lines, and
estimating the location of each of the plurality of intersection points in a three-dimensional space by triangulation based on a known distance between the intersection points when viewed from the angle of the light source.

5. The computer-based method of claim 3, wherein generating a three-dimensional model of the wound includes
identifying a first horizontal line from the plurality of horizontal lines,
estimating the location of a plurality of data points along the first horizontal line in a three-dimensional space by triangulation based on the angle of the camera relative to the light source,
identifying a first vertical line from the plurality of vertical lines, and
estimating the location of a plurality of data points along the first vertical line in the three-dimensional space by triangulation based on the angle of the camera relative to the light source.

6. The computer-based method of claim 1, further comprising:
projecting a single light line on the wound in a first location from an angle relative to a camera that captures the image of the wound;
capturing a first image of the wound with the light line in the first location;
moving the single light line to a second location on the wound; and
capturing a second image of the wound with the light line in the second location,
wherein generating a three-dimensional model of the wound includes
estimating the location of a plurality of data points along the single light line in the first image in a three-dimensional space by triangulation based on the angle of the camera relative to the light source, and
estimating the location of a plurality of data points along the single line in the second image in the three-dimensional space.

7. The computer-based method of claim 1, further comprising capturing a second image of the wound from a second camera located at an angle relative to a first camera that captured the first image of the wound, and wherein generating a three-dimensional model includes estimating a location of a plurality of data points on the wound surface in a three-dimensional space by triangulation based on the angle of the first camera relative to the second camera.

8. The computer-based method of claim 1, further comprising:
capturing a plurality of images over the period of time, and
generating a plurality of three-dimensional models of the wound based on the plurality of images.

9. The computer-based method of claim 1, further comprising generating a histogram of colors in the wound in the captured image.

10. The computer-based method of claim 1, further comprising
capturing a plurality of images of the wound over the period of time,
calculating a color density of a color in each of the plurality of images, and
measuring healing progress of the wound based on changes in the color density over the period of time.

11. The computer-based method of claim 1, further comprising
capturing a plurality of images of the wound over the period of time,
generating a plurality of three-dimensional models of the wound based on the plurality of captured images,
calculating the volume of the wound in each of the plurality of three-dimensional models, and
measuring healing progress of the wound based on changes in the volume of the wound over the period of time.

12. The computer-implemented method of claim 1, wherein calculating a volume of the wound based on the three-dimensional model includes calculating the volume of the wound under a plane located at a highest point of the wound.

13. The computer-implemented method of claim 1, wherein calculating a volume of the wound based on the three-dimensional model includes
generating an estimated healthy skin surface over the wound based on a three-dimensional model of the skin surface surround the wound, and
calculating a volume between the estimated healthy skin surface and the wound surface from the three-dimensional model.

14. A wound analysis system comprising:
a light source positioned to project at least one light line on a wound;
a first camera positioned to capture a first image of the wound at an angle relative to the light source; and
an image processing system including a processor and a computer readable memory storing computer-executable instructions that, when executed by the processor, cause the image processing system to
access the first image,
generate a three-dimensional model of the wound based on the first image,
calculate a volume of the wound based on the three-dimensional model, and
monitor changes to the calculated volume of the wound over a period of time.

15. The wound analysis system of claim 14, wherein the light source projects a plurality of parallel light lines on the wound, and wherein the computer-executable instructions, when executed by the processor, further cause the image processing system to
identify a first light line from the plurality of light lines, and
estimate the location of a plurality of data points along the first light line in a three-dimensional space by triangulation based on the angle of the first camera relative to the light source.

16. The wound analysis system of claim 14, wherein the light source projects a grid of light lines on the wound, the grid of light lines including a first plurality of parallel lines and a second plurality of lines perpendicular to the first plurality of lines.

17. The wound analysis system of claim 16, wherein the computer-executable instructions, when executed by the processor, further cause the image processing system to
identify a plurality of intersection points between the horizontal lines and the vertical lines, and
estimate the location of each of the plurality of intersection points in a three-dimensional space by triangulation based on a known distance between the intersection points when viewed from the angle of the light source.

18. The wound analysis system of claim 16, wherein the computer-executable instructions, when executed by the processor, further cause the image processing system to
identify a first line from the first plurality of parallel lines,
estimate the location of a plurality of data points along the first line in a three-dimensional space by triangulation based on the angle of the camera relative to the light source,
identify a second line from the second plurality of parallel lines, and
estimate the location of a plurality of data points along the second line in the three-dimensional space by triangulation based on the angle of the camera relative to the light source.

19. The wound analysis system of claim 14, wherein the computer-executable instructions, when executed by the processor, further cause the image processing system to
project a single light line on the wound in a first location,
receive a first image of the wound with the light line in the first location,
move the single light line to a second location on the wound,
receive a second image of the wound with the light line in the second location,
estimate the location of a plurality of data points along the light line in the first image in a three-dimensional space by triangulation based on the angle of the camera relative to the light source, and
estimate the location of a plurality of data points along the light line in the second image in the three-dimensional space.

20. The wound analysis system of claim 1, further comprising a second camera positioned to capture a second image of the wound at an angle relative to the first camera, and wherein the computer-executable instructions, when executed by the processor, further cause the image processing system to estimate the location of a plurality of data points on the wound surface in a three-dimensional space by triangulation based on the angle of the first camera relative to the second camera.

21. The wound analysis system of claim 1, wherein the computer-executable instructions, when executed by the processor, further cause the image processing system to calculate a color density of one or more colors in the captured image.

22. The wound analysis system of claim 1, wherein the computer-executable instructions, when executed by the processor, further cause the image processing system to
capture a plurality of images over the period of time, and
generate a plurality of three-dimensional models of the wound based on the plurality of images.

23. The wound analysis system of claim 22, wherein the computer-executable instructions, when executed by the processor, further cause the image processing system to
calculate a color density of one or more colors in each of the plurality of images, and
measure healing progress of the wound based on changes in the color density over the period of time.
